(19) United States
(12) Patent Application Publication
(10) Pub. No.: US 2008/0234597 A1
(43) Pub. Date: Sep. 25, 2008

(54) Monitoring Neuronal Signals

(75) Inventors: Klaus Becker, Munchen (DE); Andreas Ranft, Icking (DE); Walter Ziegiginsberger, Munchen (DE); Matthias Eder, Munchen (DE); Hans-Ulrich Dodt, Munchen (DE)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Max-Planck-Gesellschaft zur Forderung der Wissenschaften e.V., Hofgartenstrabe 8, Munchen (DE)

(21) Appl. No.: 12/067,622

(22) PCT Filed: Sep. 20, 2006
(86) PCT No.: PCT/EP2006/009160

§ 371 Date: May 21, 2008

(30) Foreign Application Priority Data
Sep. 21, 2005 (EP) 05 020 620.0

(51) Int. Cl. A61B 5/0476

(52) U.S. Cl. 600/544

(57) ABSTRACT

A monitoring device for monitoring an individual under investigation comprises a recording device for recording a neuronal signal from the individual, and a calculating device for calculating a parameter representing a quantitative measure of an anaesthesia or coma condition of the individual and being derived from the neuronal signal, wherein the calculating device is adapted for calculating a complexity parameter representing the quantitative measure of the anaesthesia or coma condition, and the calculating device includes a buffer circuit for storing at least one time series of the neuronal signal and an analysis circuit for subjecting the time series to a recurrence quantification analysis (RQA) providing the complexity parameter. Furthermore, a method includes estimating a parameter representing a quantitative measure of an anaesthesia or coma condition of an individual.

### RELATED APPLICATION

This is a §371 of International Application No. PCT/EP2006/009160, with an international filing date of Sep. 20, 2006 (WO 2007/039118 A1, published Apr. 12, 2007), which is based on European Patent Application No. 05020620.0 filed Sep. 21, 2005.

### TECHNICAL FIELD

The present application relates to a monitoring device for monitoring neuronal signals, in particular for monitoring consciousness related brain conditions like anaesthesia or coma conditions. Furthermore, the present application relates to a method of monitoring neuronal signals, in particular for calculating a complexity parameter characterizing the neuronal signals, like e.g. an anaesthetic parameter representing a quantitative measure of anaesthesia.

### BACKGROUND

Unconscious individuals do not respond to verbal command and other sensory stimulation, such as touch and pain. The clinical assessment of the cerebral (brain) function belongs to basic medical practice, and is applied e.g. for scoring trauma patients by means of the Glasgow Coma Scale. To enable major surgery, unconsciousness is induced pharmacologically by an anaesthesiologist (general anaesthesia). In case of pharmacologically induced unconsciousness, i.e., general anaesthesia for a surgical intervention, the patient's cerebral state is monitored by an anaesthesiologist.

Routine monitoring of general anaesthesia is based on clinical observation of the patient in combination with online measurement of cardiovascular parameters. More precisely, if surgical stimulation provokes neither movement nor increase in heart rate or blood pressure, it is likely that anaesthesia is sufficient. However, patients frequently receive peripherally acting muscle relaxants, preventing movement without affecting consciousness, so that the absence of movements is not significant anymore. In addition to that, in the many patients receiving betablockers, the cardiovascular response to painful stimuli is not a reliable predictor of insufficient anaesthesia either.

Moreover, in certain subgroups of patients, the dosage of anaesthetics may be restricted, such as in obstetrics or cardiac surgery, resulting in a rather “light” anesthesia. It is especially in such patients that intraoperative awareness can occur, leading to explicit memory of words spoken in the operating room, discomfort, or pain. Possible sequels of such explicit memory can be nightmares or even a posttraumatic stress disorder.

In addition to such memories accessible to conscious recall (explicit memory), implicit memories can be acquired under general anaesthesia. In this way, pain sensations, acoustic or other sensory input can influence the brain without being conscious to the individual. If the central processing of noxious stimuli is not sufficiently blocked, pain can be registered, leading to pain memory and possibly resulting in damage of nerve cells.

The right anaesthetic regimen would block such implicit memories without endangering or even harming the patient with an overdose. For this purpose, a device reliably measuring the cerebral function is required. The following techniques have been developed to derive a quantitative measure of anaesthesia from the electroencephalogram (EEG).

The raw EEG signal is processed by a Fast Fourier Transform and a power spectrum is calculated. The latter is then characterized by a number of parameters which are used to compose a single value between 0 (isoelectric EEG) and 100 (patient awake).

One of these parameters is called bispectrum index (BIS) (Sebel et a “A multicenter study of bispectral electroencephalogram analysis for monitoring anesthetic effect” in “Anesth. Analg.” vol. 84, 1997, p. 891-899). The literature about an eventual benefit of BIS-monitoring is still equivocal. While some researchers have noted a reduction of anaesthetic dosing and shorter wake-up times, others could not confirm such advantages. Cases have been documented where the BIS monitor had indicated anaesthesia in an awake patient and, vice versa, deep anaesthesia has not been recognized as such. Besides, BIS values vary depending on the drugs used, and ketamine-induced unconsciousness is not properly recognized at all.

As an alternative, an EEG signal evaluation with a combined analysis in the frequency and time domain has been developed (U.S. Pat. No. 6,011,990). Parameters obtained from the EEG signal are subjected to a multivariate classification resulting in a dimensionless index. This method has a restricted reliability as well. Like BIS monitoring, the accuracy of anaesthesia quantification depended on the anaesthetic regimen.

Another approach evaluates a multi-channel EEG for obtaining a multivariate index (so-called patient state index, PSI) (Prichep et al. “Quantitative EEG assessment of changes in the level of the sedation/hypnosis during surgery under general anesthesia” IV^{th} edition, Eds.: Jordan, Vaughan, Newton. London, Imperial College Press, 2000, pp 97-107). The drawback of this method is a low reliability and reproducibility, and a high complexity of (calculation. And again, the choice of drugs influences the result.

A. C. Watt et al. (“Anaesthesiology” vol. 81, 1994, ASA Abstract A 478) have described another approach for characterizing anaesthesia, which is based on a correspondence of a so-called EEG dimensionality and an anaesthetic level. The dimensionality is a parameter of the EEG signals obtained from a strange attractor analysis. H. C. Watt et al. have found a non-linear relationship between the dose of anaesthetic and the EEG dimensionality. However, due to low reliability and the required processing power, this approach has not been applied in medical practice.

Another method for evaluating EEG signals is the recurrence quantification analysis. As an example, N. Thomasson et al. (“Physics Letters A”, vol. 279, 2001, p. 94-101) have described the application of recurrence quantification analysis for evaluating epileptic EEG activity. With the RQA method, the detection of preictal EEG transients has been shown. As a further example, I.-H. Song et al. (“Neuroscience Letters” vol. 366, 2004, p. 148-153) have used the recurrence quantification analysis for analysing sleep EEG signals. These investigations have proved that sleep is a functional condition of the brain with high complexity being comparable with the complexity of the wake condition. Depending on the sleep phases, strong variations of EEG complexity with decreasing and increasing gradients have been found. Furthermore, the RQA method has been used for investigating a response of an organism to an external stimulus (U.S. Pat. No. 6,547,746 B1).

Generally, the conventional applications of recurrence quantification analysis of EEG signals have disadvantages in terms of a low significance of the results. Furthermore, the conventional techniques typically have allowed an off-line analysis of neuronal data only.

U.S. Pat. No. 6,442,421 B1 discloses a method for predicting epileptic seizures, wherein a current EEG is continuously compared with an EEG segment of a seizure-free phase. The comparison is based on an algorithm using data embedded in a phase space. This algorithm does not represent the RQA method. The conventional method for predicting epileptic seizures is not applicable for monitoring anaesthesia or coma conditions.

U.S. 2002/0173729 discloses a method of anaesthesia monitoring based on a calculation of a so-called approximate entropy, which does not represent the complexity parameter obtained with the RQA method. The conventional approximation may yield disadvantages in terms of significance and reproducibility of the monitoring results.

### SUMMARY

An improved monitoring device for monitoring neuronal signals is disclosed, in particular for monitoring anaesthesia or other consciousness related conditions of an individual, wherein the monitoring device is capable of avoiding the disadvantages of the conventional techniques. In particular, the monitoring device is to be improved with regard to the significance, reliability and reproducibility of monitoring neuronal signals. In particular, anaesthesia conditions are to be analysed independently on the type of anaesthesia or anaesthetic. Furthermore, an improved method of monitoring neuronal signals avoiding the disadvantages of the conventional techniques is disclosed. In particular, a method of estimating a complexity parameter.

### BRIEF DESCRIPTION OF THE DRAWINGS

Further details and advantages will be described in the following with reference to the attached drawings, which show:

FIG. 1 is a schematic illustration of a monitoring device according to an exemplary embodiment of the invention,

FIG. 2 is a schematic illustration of further details of the monitoring device according to FIG. 1,

FIG. 3 is further details of an exemplary practical embodiment of a monitoring device,

FIG. 4 is a flow diagram illustrating the steps of an exemplary method according to the invention,

FIG. 5 is a flow diagram illustrating further details of the exemplary method according to the invention,

FIG. 6 is a curve chart illustrating experimental results obtained with an embodiment of the invention, and

FIG. 7 are further curve charts illustrating experimental results obtained with an embodiment of the invention.

### DETAILED DESCRIPTION

According to a first aspect, an embodiment of the present invention is based on the general technical teaching of providing a monitoring device for monitoring an anaesthesia or coma condition of an individual under investigation, preferably for anaesthesia monitoring, comprising a recording device for recording a neuronal signal and a calculating device for calculating a complexity parameter representing a quantitative measure of a consciousness condition on the individual, wherein the complexity parameter is calculated on the basis of at least one neuronal signal time series subjected to a recurrence quantification analysis (RQA). For providing the at least one neuronal signal time series, the monitoring device includes a buffer circuit for storing neuronal signal data.

Contrary to the conventionally used narcosis monitoring techniques based on a frequency analysis of EEG data, the monitoring device according to an embodiment of the invention is adapted for an evaluation of the neuronal signals exclusively in the time domain. The inventors have found that depending on the type of anaesthetics used, frequency bands in the EEG signal can be increased or reduced. For this reason, the reliability of the conventional techniques has been found to be restricted. Contrary to the conventional concepts of characterizing a narcosis based on strange attractor theory, embodiments of the present invention provides results with relative simple data processing technique (RQA), whereby an essentially increased reproducibility has been reached.

An exemplary signal processor is disclosed being improved in terms of costs and simple structure. The application of RQA allows an essentially increased processing speed, which opens the application in practical medicine, such as in controlling surgery, The monitoring device is adapted for a reliable on-line analysis of neuronal data. The complexity parameter is obtained in real time. Further advantages of applying RQA are derived from the fact that RQA is implemented on the basis of a set of RQA parameters, which can be directly adjusted at the monitoring device. The RQA applied yields a complexity parameter which can be directly presented e.g. on a display and which can be used as a direct quantitative measure of the consciousness condition, e.g. anaesthesia condition of the individual under investigation.

Furthermore, contrary to the conventionally used techniques based on a spectral analysis of EEG signals, the complexity parameter obtained is independent on the anaesthetic used. The RQA can be applied in particular with high reproducibility with anaesthesias based on Ketamin, Propufol, Sevofluran. This anaesthetic type independency represents an essential advantage compared with the conventional techniques as the monitoring device allows a precise control of short-time narcosis treatments, in particular for ambulant surgery or with shallow narcosis treatments, e.g. with hearth surgery or births.

As used herein, the term “anaesthesia” refers to a total or partial loss of sensation induced by disease or an anaesthetic, while the term “coma” refers to a deep, prolonged unconsciousness, e.g. induced by an injury, a disease or poison. Accordingly, the complexity parameter can be used for characterizing the depth of anaesthesia or coma conditions. The term “complexity parameter” refers to a quantity (preferably a single numeric value, e. g. in the range of 0 to 1 or 0% to 100%) being a statistic measure of the randomness or probability of physiological (neuronal) processes determining an anaesthesia or coma condition.

According to a further aspect, the monitoring device can be provided as compact equipment for routine operation in medical practice. The compact construction as a combination of standard components yields an essential cost reduction compared with conventional devices having costly spectral analysers. Furthermore, the monitoring device may be adapted to requirements in medical applications in terms of easy use, cleaning and disinfection.

A further aspect is related to the fact that the monitoring device can be adapted for processing various types of neuronal signals, which comprise any type of electro-physiological data, which in particular can be recorded with conventional techniques. As examples, the neuronal signal may comprise EEG signals, EMG (electro myography) signals, or signals measured with the patch-clamp technique or derived from electro-physiological field potentials. According to an exemplary embodiment, the monitoring device is adapted for processing EEG signals. This embodiment has particular advantages in terms of the available technology of EEG recording. EEG measurements represent a standard tool during surgery. Accordingly, the recording device used may comprise simply an EEG recorder, which can be combined with a standard EEG sensor equipment.

According to another exemplary embodiment, the EEG recorder is adapted to record one EEG signal only. Accordingly, the EEG recorder is connected with exactly two signal lines (measurement line/reference line) only. The inventors have found that one revulsion of electro-physiological data is enough for implementing the RQA according to the invention. The monitoring device does not necessarily require the recording of a complete EEG with e.g. 2 or more revulsions. Additionally, a ground line may be used for avoiding influences from the power supply system.

According to another exemplary embodiment, the monitoring device is adapted for evaluating the EEG signal in a predetermined frequency band only. For this purpose, the EEG recorder or the calculating device includes a filter circuit for recording or processing EEG data having frequencies in the selected frequency band. This embodiment has an advantage as data processing can be adapted to a frequency band with high contents of relevant information. Particularly preferred is the recording of EEG signals in the gamma-frequency band, which typically is not influenced by sleep effects. According to this embodiment, RQA is implemented in another frequency band compared with the conventional investigations of sleep data. According to a further exemplary embodiment, frequency components with frequencies above the gamma frequency band are recorded. The inventors have found that in the high-frequency range (above 20 Hz), complexity parameters can be obtained with high reproducibility and low signal-to-noise-ration (SNR).

The monitoring device according to an exemplary embodiment includes an analysis circuit being adapted for implementing the recurrence quantification analysis. Depending on the application of the monitoring device, the analysis circuit can be provided with a fixed set of RQA parameters yielding an advantage in terms of simple structure of the monitoring device and increased processing speed. However, according to an exemplary embodiment, the analysis circuit is adapted for setting RQA parameters. This embodiment allows an improved adaptation of the monitoring device to the practical application. Preferably, the analysis circuit includes an adjustment device for setting the RQA parameters. The adjustment device can be adapted for manually setting the RQA parameters by an operator or for automatically setting the RQA parameters in dependence on the operation conditions of the monitoring device. In the latter case, preferably a control loop is implemented for controlling of the adjustment device in dependence on the output of the calculating device.

According to a further exemplary embodiment, the calculating device may include at least one of an evaluating device for further processing the complexity parameter and an alarm device for indicating predetermined alarm conditions of the complexity parameter or any other value obtained from the complexity parameter by further processing.

According to a second general aspect, an anaesthesia device is provided for surgery anaesthesia, which includes the above monitoring device. Furthermore, the anaesthesia device includes an anaesthesia control device for controlling anaesthesia parameters, like the type of anaesthetic or the time of supplying a certain anaesthetic. The anaesthesia device has an essential advantage in terms of being suitable for an automatic or semi-automatic anaesthesia. In this case, the anaesthetist is required for monitoring the anaesthesia device only, while essential functions, like e.g. dosing the anaesthetic is provided automatically.

According to a further general aspect, various embodiments of the present invention may be based on the general technical teaching of providing a method of estimating a parameter representing a quantitative measure of anaesthesia or coma, wherein time series of a neuronal signal from an individual under investigation are subjected to a recurrence quantification analysis yielding a complexity parameter. RQA has been found as being a significant tool for determining the regularity or complexity of time series of neuronal signals.

Basic principles and theory of RQA have been developed and published by C. L. Webber et al. in “J. Appl. Physiol.” vol.76, 1994, p. 965-973. The time series with a predetermined number of values are represented in a phase space with a predetermined embedding dimension m. The variation of the neuronal signal, in particular of a recorded EEG signal yields a trajectory of the current state in the phase space. The complexity parameter measured with RQA is a quantitative characterization of the trajectory movement in the phase space. Strong variations of the trajectory represent a high complexity, while low variations represent a low complexity. The variation of the trajectory or a value derived from this variation can be used for a direct classification of the complexity parameter.

The present inventors have found preferred ranges of the RQA parameters embedding dimension (m), time delay (d), matrix threshold (r) and minimal line length (L). According to an exemplary embodiment, the embedding dimension m (number of values of each time series) is selected to be in the range of 10 to 30. This embedding dimension range represents advantages in terms of processing speed and reproducibility. Furthermore, the parameter time delay used for the subsequent construction of time series may be preferably selected in the range of 1 to 20. With matrix threshold (r) in the range of 0,1 to 0,8, e. g. 0,1 to 0,6, a suitable sensitivity in particular for anaesthesia monitoring has been obtained. Minimal line length (L) in the range of 3 to 500, in particular 50 to 500, has been proved to result in high stability and reproducibility.

Another independent aspect is represented by the application of the monitoring device or the method for long-term monitoring brain functions of a patient or for monitoring and/or controlling anaesthesia induced by an anaesthetic during a surgical operation.

An exemplary embodiment is described in the following with reference to anaesthesia monitoring during surgery. Details of medical equipment used for EEG recording and anaesthesia are not described as far as they are known from conventional medicine techniques. Furthermore, it is emphasized that the invention can be implemented in an analogue way with alternative applications for monitoring anaesthesia induced by a disease or monitoring coma patients.

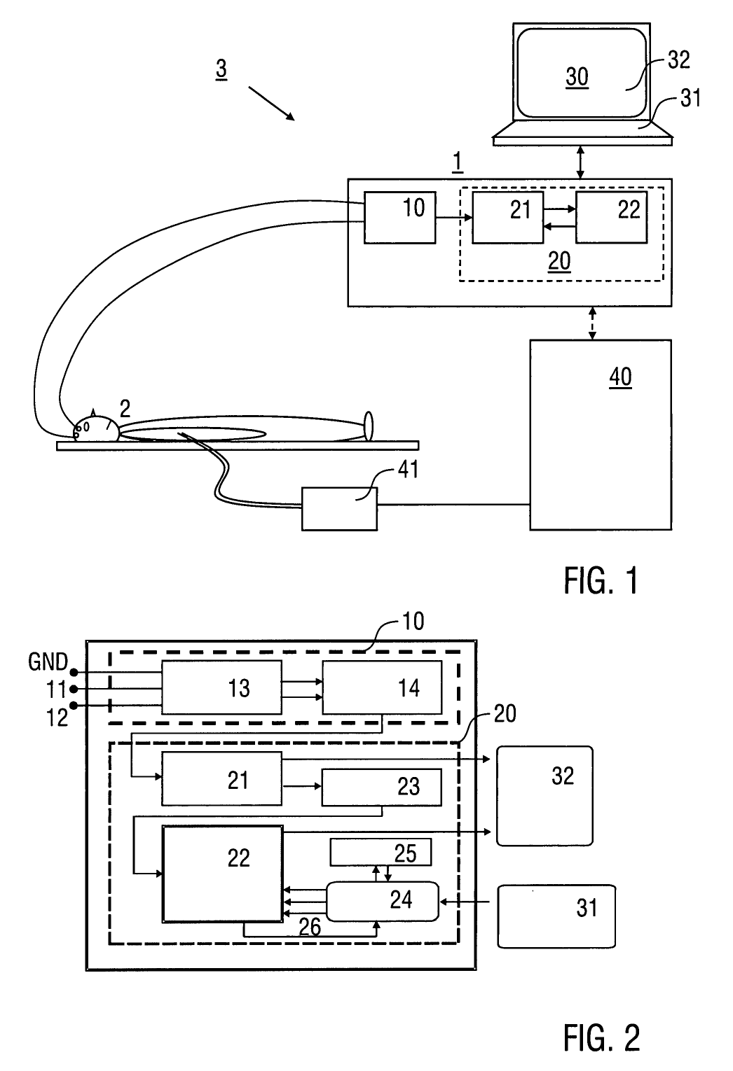

FIG. 1 schematically illustrates an embodiment of the monitoring device 1 with an EEG recorder 10 and a calculating device 20 including a buffer circuit 21 for storing at least one time series of EEG signals and an analysis circuit 22 for analysing the EEG signal with recurrence quantification analysis. The EEG recorder has a structure like a conventional recorder device. The calculating device 20 comprises a standard board with processors implementing the calculation and adjustment functions outlined below.

The EEG recorder 10 is connected via two signal lines 11, 12 with EEG sensors to be applied on the head of an individual 2 under investigation. The lines comprise a measurement line and a reference line. Additionally, a ground line is provided. The monitoring device 1 is equipped with a control unit 30 including an input interface 31 and a display 32. Components 10, 20 and 30 are arranged within a common casing. The complete anaesthesia device 3 according to the invention comprises the monitoring device land an anaesthesia control device 40 controlling an anaesthetic supply unit 41.

Further details of the monitoring device 1 are illustrated in FIG. 2. The EEG recorder 10 comprises an EEG amplifier 13 and an A/D-converter 14. The input of the EEG amplifier is connected with the signal lines 11, 12 connected with a signal electrode and a reference electrode on the patient as well as with a ground electrode. EEG signals amplified with the EEG amplifier 13 are digitised with the A/D-converter 14. The digital EEG signals are output to the calculating circuit 20.

The calculating circuit 20 comprises the buffer circuit 21, the analysis circuit 22, the filter circuit 23 and an adjustment circuit 24. These components are arranged on e dual processor computer main board. The digital EEG data are stored in the buffer circuit 21. The filter circuit 23 comprises a digital FIR-filter. EEG data having frequency components in a predetermined frequency range of e.g. 20 Hz to 150 Hz are delivered via the filter circuit 23 to the analysis circuit 22, which is adapted for the calculation of the complexity values as outlined below. This calculation comprises the recurrence quantification analysis implemented with certain RQA parameters, which are supplied from the adjustment circuit 24 to the analysis circuit 22. The RQA parameters can be stored in a further parameter memory 25 or set by an operator via the input interface 31, e.g. by using optical encoders. Furthermore, a control loop 26 can be implemented for varying RQA parameters in dependence on the result of the complexity value calculation. The raw data stored in the memory circuit 21 and the complexity parameter calculated in the analysis circuit 22 are output to the control unit and displayed on the display device 32, which comprises e.g. a LCD display.

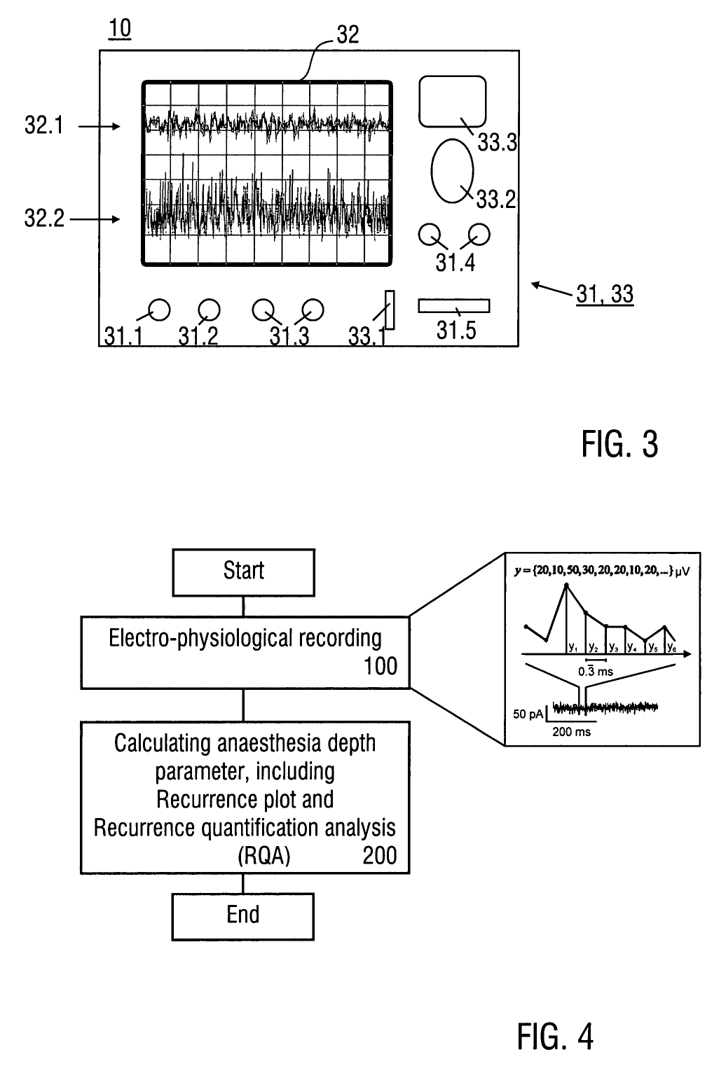

The components 10, 20 and 30 shown in FIG. 1 are preferably provided in a single compact device. An example of a front panel of a compact monitoring device 10 is illustrated in FIG. 3. The front panel includes the input interface 31 and the display 32 as shown in FIG. 1. The input interface 31 comprises a power switch 31.1, a radius adjustment element 31.2, scaling elements 31.3 for scaling the traces 32.1 and 32.2 of the display 32, and velocity trace elements 31.4. In the upper trace 32.1 represented in display 32, the raw data of the EEG signal are shown, while the lower trace 32.2 represents the calculated complexity parameters.

Additionally, the front panel serves as an output interface 33 with a digital data output 33.1, loud speaker 33.2 connected with an alarm device and a numerical representation 33.3 e.g. of the actual complexity parameter and a further reference parameter, such as e.g. an average complexity parameter. The reference numeral 31.5 refers to the connector for accommodating the signal and ground lines 11, 12 and GND illustrated in FIG. 2.

The operation principles of the exemplary monitoring device are described in the following with exemplary reference to the calculation of an anaesthesia depth parameter (complexity parameter) as shown in FIGS. 4 to 6. According to FIG. 4, an exemplary method comprises the steps of electro-physiological recording of a neuronal signal, in particular an EEG signal (step 100) and the step of calculating an anaesthesia depth parameter on the basis of RQA (step 200).

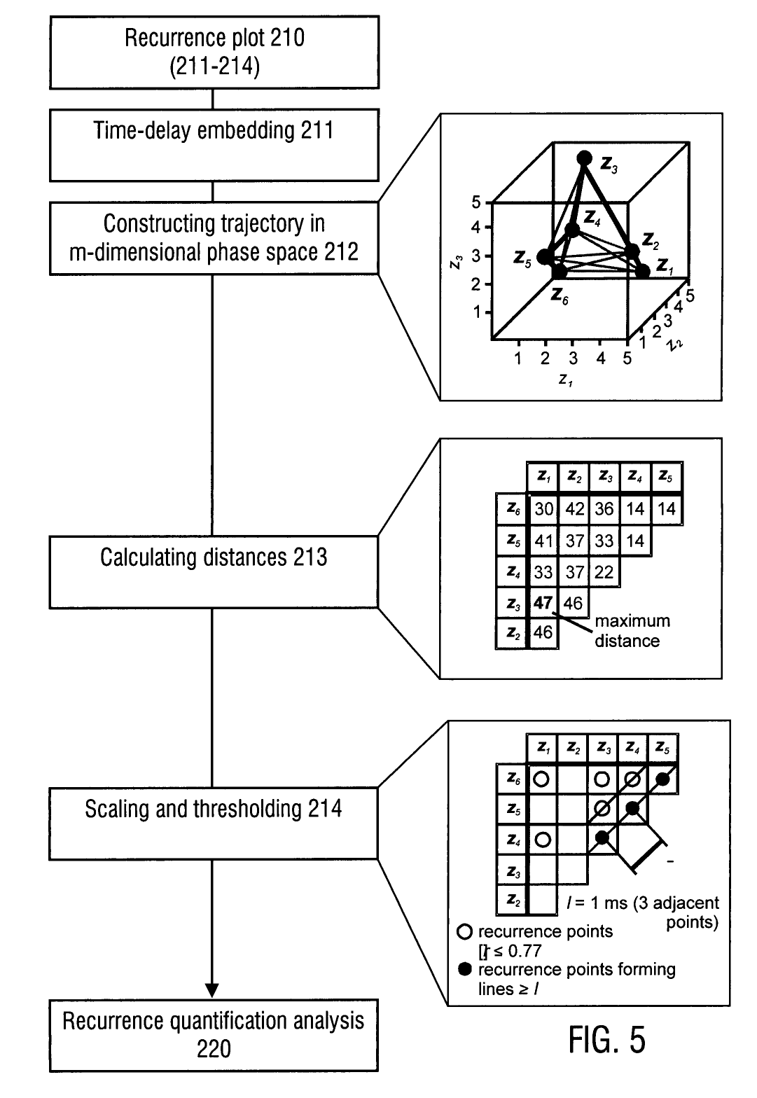

With step 100, time series of discrete EEG voltage or current values are recorded. Each time series represents a segment of measured values with a length of e.g. 1000 samples. The time series (segments) are stored in the memory circuit 21 (see FIG. 2) without overlap in time. The sample frequency is selected in the range of 500 Hz to 2 kHz, e.g. 1 kHz. The insert of FIG. 4 illustrates the measurement of current values. The measured values being sampled with a time interval of 1 ms yield the time series y indicated in the upper part of the insert. The following calculating step 200 comprises the recurrence plot 210 with sub-steps 211 to 214 and the recurrence quantification analysis 220 as illustrated in FIG. 5.

As the first sub-step 211, the recorded time series are subjected to time-delay embedding. Time-delay embedding comprises a substitution of each measured value by a vector with the embedding dimension m. According to the theorem of Takens, the embedding vector Z comprises the components zᵢ according to:

zᵢ=y_{b}yᵢ+τ, . . . , yi+(m−1)τ

with m being the embedding dimension and τ being the time delay parameter.

While embedding dimension m used in practice is preferably selected with e. g. m=10 or m=20, the present illustration refers to the parameters m=3 and τ=0.3 ms for clarity reasons, although it should be understood that other parameters may be selected as well where desired. With these parameters, the measured values are replaced by a series of time delay vectors:

$$z = \{ ( \begin{matrix}50 \\ 10 \\ 20\end{matrix} ) \cdot ( \begin{matrix}30 \\ 50 \\ 10\end{matrix} ) \cdot ( \begin{matrix}20 \\ 30 \\ 50\end{matrix} ) \cdot ( \begin{matrix}20 \\ 20 \\ 30\end{matrix} ) \cdot ( \begin{matrix}10 \\ 20 \\ 20\end{matrix} ) \cdot ( \begin{matrix}20 \\ 10 \\ 20\end{matrix} ) \cdot … \} μ V$$

Each vector can be considered as representing a specific point in the phase space. As the next sub-step, the time-delay vectors are used for constructing a trajectory in the m-dimensional phase space (step 212 in FIG. 5). The sequence of vectors forms a trajectory as exemplary illustrated in the upper insert of FIG. 5. The trajectory is characterized by a random variation (“jumping”) in time. The RQA applied according to an exemplary embodiment is used for quantitatively characterizing the degree of variation of the trajectory in phase space, which is considered as the complexity parameter for describing the consciousness condition of the brain during anaesthesia or coma.

Subsequently, the distances of the points in phase space are calculated with the next sub-step 213. The distances of all points zᵢ relative to each other are estimated and represented in a triangular matrix (table) as shown in the insert of FIG. 5. According to a first alternative, the distances are calculated with the euclidic metric:

$$dist ( {z}_{i} , {z}_{j} ) = \sqrt{{\sum }_{k = 1}^{m} {( {z}_{i , k} - {z}_{j , k} )}^{2}}$$

According to a second alternative, the “city-block”—or Manhattan-metric is used for calculating the distances according to:

$$dist ( {z}_{i} , {z}_{j} ) = {\sum }_{k = 1}^{m} ( {z}_{i , k} - {z}_{j , k} )$$

The euclidic metric has an advantage in terms of precise calculation of the distances. However, the “city-block”—metric is preferred as the inventors have obtained results with high precision (comparable with euclidic metric) while the processing speed is increased and the processing costs is essentially reduced.

With the next sub-step 314, a scaling and thresholding is implemented. Scaling comprises a standardisation of the calculated distances. According to a first alternative, the standardisation is implemented relative to an average distance (so-called average scaling). According to a second alternative, the standardisation is implemented relative to a maximum distance obtained in phase space (so-called maximum-scaling). For calculating complexity parameters according to the invention, the application of maximum-scaling is preferred. As a result, scaled distances in the range of 0 to 1 are obtained. With the illustrated example, all elements of the triangular matrix are divided by 47.

According to a further alternative, standardisation can be omitted in step 314. In this case, the further calcultation is implemented directly with the distance measure (ms).

Subsequently, all scaled distance values are subjected to a matrix thresholding which comprises a digitalisation wherein all scaled distance values equal or above a predetermined threshold value r are set to 1, while all remaining distance values below r are set to 0.

The matrix threshold value r is selected depending on the particular application. For evaluating EEG signals, r=0.4 has been found to be suitable. Preferably, the threshold value is adjustable for optimising the sensitivity of the method to the requirements of a particular application.

The result of the matrix thresholding of scaled distance values is illustrated in the insert (bottom) of FIG. 5. All points below the matrix threshold value r (set to 0) are illustrated with circles while the other values (set to 1) are omitted. The graphical representation illustrated in the insert (bottom) of FIG. 5 represents a recurrence plot, which in practice has an essentially more complex appearance.

On the basis of the recurrence plot, the recurrence quantification analysis (step 220) is implemented as follows. The recurrence plot includes points forming diagonal lines (full circles). The length of these lines is characteristic for time ranges in which the trajectory of the system has low variations only. In these time ranges, the system has a low complexity or dynamic (relative stable condition). The RQA comprises a further thresholding with a so-called minimal line length parameter L (minimal line length thresholding). All points in the recurrence plot forming a diagonal line with a length greater than L are estimated. In practice a minimal line length parameter L=100 is preferred.

Finally, the ratio of points fulfilling the minimal line length thresholding condition and the complete number of points in the recurrence plot (fulfilling the above scaled value thresholding condition) is calculated. The complexity parameter to be obtained with the method of the invention is represented by the determinism parameter D [%] according to:

$$D = \frac{number of recurrence points forming lines \geq I}{total number of recurrence points} \times 100$$

Determinism D is a quantitative measure for the predictability or regularity of the time series measured. For the application in anaesthesia monitoring, the calculation of the complexity K according to

K=−log(D)

is preferred. The value K=0 would represent a perfect regularity, while higher values indicate a more complex behaviour. For providing a complexity parameter K′ with dB scale, K′ is calculated as −20 log(D).

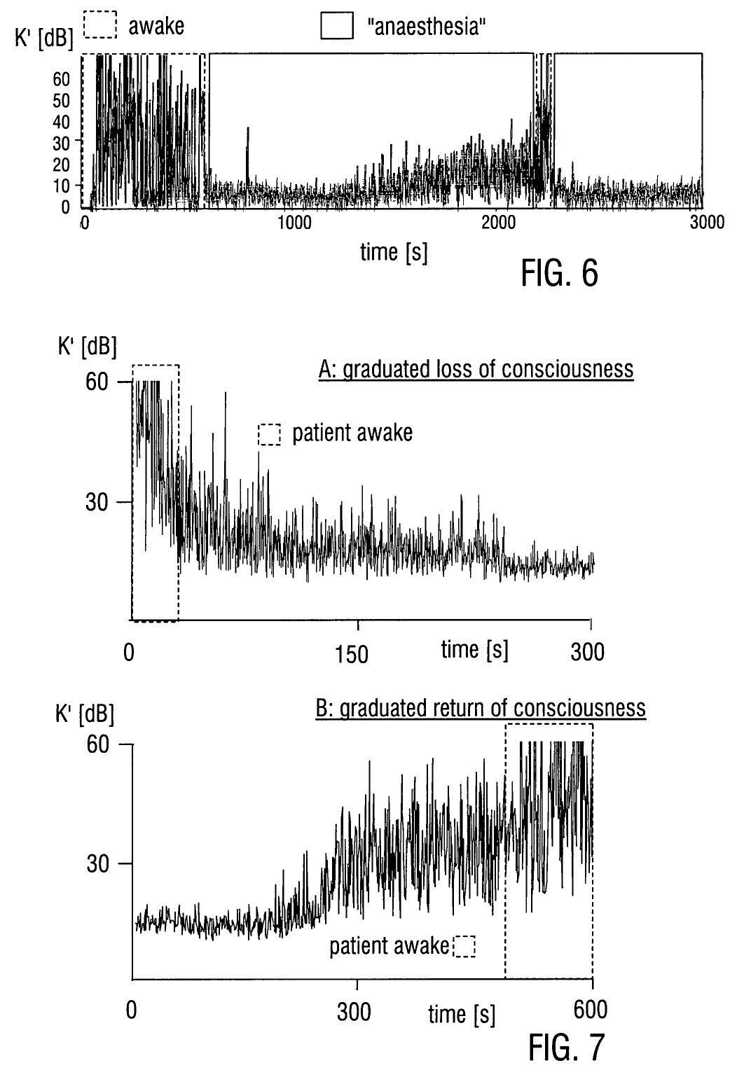

FIG. 6 illustrates experimental results of RQA with EEG signals. The inventors have found various phases with high D values and essentially reduced D values, respectively. These different phases (“awake” and “anaesthesia”) have been correlated with different conditions of a patient obtained with monitoring vegetative parameters of the individual indicating awake and anaesthesia conditions corresponding to high and low D values, respectively.

The results of the inventors have shown that the parameter K has a pronounced dependency on time. After injection of an anaesthetic, the K value is decreased within a range of 1 to 2 min down to a constant value (see FIG. 7A). This K gradient in time has been found with high reproducibility. On the other hand, the K value is increased after completing the anaesthetic supply within a range of about 10 to 20 min (see FIG. 7B). After this phase of increasing the K value, a K value jump has been found being correlated with the wake-up of the individual.

The reproducible time gradients with decreasing and increasing K values and the reproducible K value jump allow a calibration of the K value profiles obtained with an individual. On the basis of this calibration, a quantitative measure for narcosis depth has been found, which is reproducible and suitable for describing various phases of anaesthesia.

On the basis of the anaesthesia parameter obtained with the invention, the anaesthesia control device 40 (FIG. 1) can be set for supplying the anaesthetic supply 41 for obtaining a predetermined degree of narcosis.

The features of the invention disclosed in the above description, the drawings and the claims can be of significance both individually as well as in combination for the realization of the invention it its various embodiments.

1.-22. (canceled)

23. A monitoring device for monitoring an individual under investigation, comprising:
a recording device for recording a neuronal signal from the individual, and
a calculating device for calculating a parameter representing a quantitative measure of an anaesthesia or coma condition of the individual and being derived from the neuronal signal, wherein
the calculating device is adapted for calculating a complexity parameter representing the quantitative measure of the anaesthesia or coma condition, and
the calculating device includes a buffer circuit for storing at least one time series of the neuronal signal and an analysis circuit for subjecting the time series to a recurrence quantification analysis (RQA) providing the complexity parameter.

24. A monitoring device according to claim 23, wherein the recording device includes an EEG recorder for recording an EEG signal being the neuronal signal from the individual.

25. A monitoring device according to claim 24, wherein the EEG recorder includes two signal lines to be connected with the individual.

26. A monitoring device according to claim 24, wherein the EEG, recorder or the calculating device includes a filter circuit for recording frequency components in a predetermined frequency band of the EEG signal.

27. A monitoring device according to claim 26, wherein the filter circuit is adapted for recording frequency components in the gamma-frequency band of the EEG signal.

28. A monitoring device according to claim 23, wherein the analysis circuit includes an adjustment device for setting RQA parameters of the RQ analysis.

29. A monitoring device according to claim 28, wherein the calculating device includes a control loop for controlling the adjustment device.

30. A monitoring device according to claim 23, wherein the calculating device includes an evaluation device for further processing the complexity parameter.

31. A monitoring device according to claim 30, wherein the evaluation device includes an alarm device.

32. An anaesthesia device comprising a monitoring device according to claim 23.

33. A method of estimating a parameter representing a quantitative measure of an anaesthesia or coma condition of an individual, comprising the steps of:
recording of a neuronal signal from an individual under investigation, and
calculating the quantitative measure of the anaesthesia or coma condition, wherein
the recording step includes the step of storing at least one time series of the neuronal signal, and
the calculating step includes the step of subjecting the time series to a recurrence quantification analysis (RQA) for providing a complexity parameter being derived from the neuronal signal.

34. A method according to claim 33, wherein the neuronal signal comprises an EEG signal and the recording step includes the step of recording the EEG signal from the individual.

35. A method according to claim 34, wherein frequency components in a predetermined frequency band of the EEG signal are recorded.

36. A method according to claim 35, wherein frequency components in the gamma-frequency band of the EEG signal are recorded.

37. A method according to claim 33, wherein the stored time series of the neuronal signal comprises at least 500 signal values.

38. A method according to claim 33, further comprising the step of setting at least one RQA parameter, wherein the RQA parameter comprises at least one embedding dimension (m), time delay (d), matrix threshold (r) and minimal line length (L).

39. A method according to claim 38, wherein at least one of the RQA parameters is set in the following ranges:
embedding dimension (m) in the range of approximately about 10 to about 30.
time delay (d) in the range of approximately about 1 to about 20 s,
matrix threshold (r) in the range of approximately about 0,1 to about 0,8, and
minimal line length (L) in the range of approximately about 3 to about 500.

40. A method according to claim 33, wherein the RQA includes a step of calculating distances of phase space vectors on the basis of a city-block-metric.

41. A method according to claim 33, wherein the RQA includes a step of normalizing the calculated distances relative to a maximum distance value.

42. A method according to claim 33, wherein the complexity parameter comprises a complexity value K obtained from an RQ determinism value D according to
K=−log D.

43. A method according to claims 33, further comprising the step of evaluating the complexity parameter.

44. A method of using a monitoring device or a method as in any one of the preceding claims, for:
long term monitoring brain functions of a patient, or
at least one of monitoring and controlling anaesthesia during a surgical operation.
